(19) United States
(12) Patent Application Publication
(10) Pub. No.: US 2018/0360861 A1
(43) Pub. Date: Dec. 20, 2018

(54) PRODUCT AND METHOD FOR TREATMENT OF BIOFILM, INCLUDING CONTROL OF SUBSTRATE COLONIZATION AND TREATMENT OF INFECTION

(71) Applicant: Reduxx LLC, Fort Wayne, IN (US)

(72) Inventors: Carl Hilliard, San Diego, CA (US); William R. Cast, Fort Wayne, IN (US)

(73) Assignee: Reduxx LLC, Fort Wayne, IN (US)

(21) Appl. No.: 16/058,644

(22) Filed: Aug. 8, 2018

Related U.S. Application Data
(63) Continuation of application No. 14/716,589, filed on May 19, 2015, now abandoned.
(60) Provisional application No. 62/000,403, filed on May 19, 2014.

(51) Int. Cl. A61K 31/695; A61L 31/16; A61K 47/20; A61K 45/06; A61K 9/50; A61L 15/20; A61L 15/44

(52) U.S. Cl. CPC A61K 31/695; A61L 2300/622; A61L 2300/404; A61L 31/16; A61K 2300/00; A61K 45/06; A61K 9/50; A61L 15/20; A61L 15/44; A61K 47/20

(57) ABSTRACT

An antimicrobial product and methods of use are provided. The antimicrobial product includes a water-soluble antimicrobial organosilane and various additional compound, including antibiotic and anti-inflammatory medications, antiseptics, and/or detergents. An example delivery system including a microcapsule encasing the product is also described. Methods of use include embedding a delivery system within an article, such as bandages, inserts or wound coverings, the microcapsule containing the product which is released by a mechanism and at a time particular to the intended use of the article. The methods include treating an infection on a substrate by disrupting an existing microbial colonization, penetrating existing biofilms, and/or killing existing microbes.

### CROSS REFERENCE TO RELATED APPLICATIONS

This application is a continuation application of U.S. patent application Ser. No. 14/716,589, filed May 19, 2015, which claims priority to U.S. Provisional Patent Application No. 62/000,403, filed May 19, 2014, the entirety of which are incorporated herein by reference for all purposes.

### BACKGROUND

### Technical Field

This disclosure relates generally to antimicrobial compounds with antimicrobial activity; in particular, to organosilane compounds for treatment of biofilms and other microbial pathogens in animals and humans, controlling colonization of wounds and epithelial surfaces, and treatment of infections involving same.

### State of the Art

Prevention and treatment of infection in humans and animals has been a public health goal since the discovery of microorganisms and their role in causing disease. As an outgrowth of the germ theory of disease, much progress has been made in controlling the spread, dissemination, and effects of pathogenic microorganisms. For example, simple techniques such as routine hand-washing and thorough cleaning of hard surfaces are highly effective in preventing the spread of diseases which are disseminated by contact. When infection occurs despite such precautions, treatment with topical and systemic antimicrobials, such as the use of antibiotics, are valuable adjuncts to these preventive measures. Despite these advances, however, microbial infections remain the number one cause of death globally. A biofilm may be formed by particles, organic or inorganic, that flow or settle onto the surface, including layers of dead microorganisms, their products and detritus “the Conditioning Layer.” The Conditioning Layer facilitates the adhesion of planktonic microbes to the substrate and provides nutrients and proximate pathogens that aid in the growth and defense of the colony. Upon attachment to the Conditioning Layer, microbes begin to divide into new cells and the three dimensional biofilm begins to take shape. Up to forty percent (40%) of the genes of the individual microbes change status as they assume specialized functions during the transition from planktonic to biofilm state. Inter cellular communications are established between the cells within the biofilm through a chemical means called “quorum sensing.” These pronounced physiological changes help biofilms resist disinfection, develop antibiotic resistance, release deadly toxins and break down materials. Because of the decreased permeability of the outer layer of thick biofilms to various compounds, a surface covered with a biofilm is more resistant to disinfection. Biofilms may survive under harsh conditions, and the embedded cells may be up to 1000 times less susceptible to disinfectants and biocides. An increased rate of exchange of genetic material between individual microbes of different bacterial or fungi types that are densely packed together has been demonstrated in biofilms. Such exchange is thought to promote mutations and build up biofilm defenses. Wherein the exchanged genetic material confers additional resistance to an antibiotic this process leads to the rapid establishment of antibiotic resistance within the biofilm's contiguous population of pathogens. Accordingly, the control of microbial growth is an important issue in the broad field of science as well as medicine.

Biofilms and other pathogens, however, create challenges for preventing and treating microbial infections on the skin and subcutaneous tissues of animals and humans. Microorganisms, including bacteria, viruses, and fungi, are present in the environment and can be spread from carrier to carrier through physical contact. Depending on the microorganism, the carrier, and ambient environmental conditions, microbial cells on an open wound or epithelial surface may proliferate, eventually resulting in formation of a biofilm. Biofilms resist disinfection, develop antibiotic resistance, and break down materials. Because of the decreased permeability of thick biofilms to various compounds, a substrate infected with a biofilm is more resistant to disinfection. Biofilms may allow microbial cells to survive under harsh conditions, and the embedded cells may be up to 1000 times less susceptible to disinfectants and biocides. An increased rate of exchange of genetic material between individual microbes densely packed together has been demonstrated in biofilms. Wherein the exchanged genetic material confers resistance to an antibiotic this process leads to the rapid establishment of antibiotic resistance within the biofilm's contiguous population of bacteria. Accordingly, the control of microbial colonization of substrates, including effective treatment of colonization and invasive infections involving biofilms, is an important issue in the fields of veterinary and human medicine. Compounds with lower or diminished levels of concentration have been discarded into the environment as waste allowing pathogens coming into contact with such diluted compounds to withstand their effect and become resistant to such antibiotics.

One way to prevent surface contamination is to sterilize the local environment by using disinfectants. Disinfectants act by denaturing cellular proteins, breaking nucleic acid chains, and disrupting bacterial cell walls. Disinfectants on skin or subcutaneous tissues, however, wash off and must be replaced at least daily. Further, disinfectants have been shown to select for resistant antimicrobial strains; e.g., methicillin resistant Staphylococcus aureus (MRSA), which causes dangerous nosocomial infections and results in more deaths in the United States than HIV/AIDS.

Another way to prevent substrate contamination is to treat the wound of skin surface with an antimicrobial product to create an antimicrobial coating that is resistant to microbial growth over a long period of time. These treated surfaces are coated with more concentrated agents, which, depending on the agent used, may be toxic to the animal or human.

The reliance on antibiotics has resulted in microbial adaptation resulting in the creation of “superbugs” that are resistant to current clinical treatments. In this regard, it can be appreciated that there is a critical need for compounds that can be applied topically to clear infections without encouraging increased microbial resistance.

### DISCLOSURE OF EMBODIMENTS OF THE INVENTION

The present disclosure relates generally to antimicrobial compounds with antimicrobial activity; in particular, to organosilane compounds for disruption of the formation of a biofilm, interfering with the communication between cells within a biofilm and interruption with the homeostasis of its matrix for treatment of infections of skin and subcutaneous tissues in animals and humans, controlling colonization of wounds and epithelial surfaces, and treatment of other pathogens found in infections involving same.

Disclosed is an antimicrobial product comprising an organosilane; a carrier; and a delivery system.

In some embodiments, the delivery system is a microcapsule enclosing the organosilane therein. In some embodiments, the organosilane is a 3-(trihydroxysilyl) quaternary ammonium compound, such as, but not limited to such as 3-(trihydroxysilyl) propyl dimethyl octadecyl ammonium chloride. In some embodiments, the concentration of the organosilane is less than 0.10 percent by weight. In some embodiments, the concentration of the organosilane is between 0.10 percent and 1.00 percent by weight. In some embodiments, the concentration of the organosilane is greater than 1.00 percent by weight. In some embodiments, the concentration of the organosilane is greater than 5.0 percent by weight. In some embodiments, the carrier is a compound selected from the group of carrier compounds consisting of: an alcohol, a wax, or dimethyl sulfoxide.

In some embodiments, the product, further comprises an enzyme. In some embodiments, the enzyme is a proteolytic hydrolase enzyme. In some embodiments, the enzyme is an enzyme acting upon a substrate comprising N-acyl homoserine lactone

In some embodiments, the product further comprises a detergent. In some embodiments, the detergent is a quaternary ammonium compound. In some embodiments, the product further comprises an antibiotic molecule. In some embodiments, the antibiotic molecule is a compound selected from the group of antibiotic molecules consisting of: an aminoglycoside, a macrolide, ciprofloxacin, polymyxin B, or a sulfonamide. In some embodiments, the antimicrobial product further comprises an anti-inflammatory. In some embodiments, the anti-inflammatory comprises a steroid molecule. In some embodiments, the anti-inflammatory is a compound selected from the group of anti-inflammatory compounds consisting of: hydrocortisone, triamcinolone diacetate, beta methasone valerate, beta methasone diproprionate, resorcinol, and methyl resorcinol. In some embodiments, the antimicrobial product further comprises an antiseptic. In some embodiments, the antiseptic is a compound selected from the group of antiseptic compounds consisting of: benzethonium chloride, benzalkonium chloride, sodium oxychlorosene, hypochlorous acid, hexylresorcinol, methyl resorcinol, poloxamer iodine complex, iodine complex, secondary amyltricresols, and ethyl alcohol. In some embodiments, the antimicrobial product further comprises a topical anesthetic. In some embodiments, the topical anesthetic is a compound selected from the group of topical anesthetic compounds consisting of: lidocaine hydrochloride, hexylresorcinol, methyl resorcinol and benzocaine hydrochloride. In some embodiments, the antimicrobial product further comprises a keratolitic. In some embodiments, the keratolitic agent is salicylic acid. In some embodiments an agent is added to increase penetration and absorption. In some embodiments this penetrating agent is DMSO, dimethyl sulfoxide.

The antimicrobial product of claim 2, wherein the antimicrobial product further comprises a buffer. In some embodiments, the buffer is a compound selected from the group of buffer compounds consisting of: a citrate, a sulfonate, a carbonate, and a phosphate.

Disclosed is a method of providing an antimicrobial treatment to a substrate, the method comprising the steps of applying a product comprising an organosilane to a substrate; and adhering the organosilane to the substrate.

Disclosed is a method of treating an infection, the method comprising steps of applying an antimicrobial product comprising an organosilane to a substrate; killing a microbial cell; and establishing a mechanical barrier against colonization of the substrate by additional microbial cells.

In some embodiments, the method further comprises a step penetrating a biofilm.

In some embodiments, the method further comprises a step of placing treated material in proximity to an area of microbial colonization.

The foregoing and other features and advantages of the present invention will be apparent from the following more detailed description of the particular embodiments of the invention, as illustrated in the accompanying drawings.

### BRIEF DESCRIPTION OF THE DRAWINGS

Some of the embodiments will be described in detail, with reference to the following figures, wherein like designations denote like members:

FIG. 1 is a schematic diagram showing a general chemical structure of an organosilane molecule;

FIG. 2 is a schematic diagram showing a general chemical structure of an organosilane molecule;

FIG. 3 is a schematic representation showing organosilane molecules adhered to a substrate in the presence of microbial cells;

FIG. 4 is a schematic representation of a delivery system comprising a microcapsule for an antimicrobial product;

FIG. 5 is a diagram of a method 200 of treating infection and/or infectious disease and/or providing continuing protection against re-infection during the remaining life of the substrate;

FIG. 6 is a diagram of a method 300 of treating infection and/or infectious disease and/or continuing protection against re-infection during the remaining life of the substrate; and

FIG. 7 is a diagram of a method 500 of treating and preventing an infection on a substrate.

### DETAILED DESCRIPTION OF EMBODIMENTS OF THE INVENTION

A detailed description of the hereinafter described embodiments of the disclosed method are presented by way of example and not meant to be limiting with reference to the Figures listed above. Although certain embodiments are shown and described in detail, it should be understood that various changes and modifications may be made without departing from the scope of the appended claims. The scope of the present disclosure will in no way be limited to the number of constituting components, the materials thereof, the shapes thereof, the relative arrangement thereof, etc., and are disclosed simply as an example of embodiments of the present disclosure.

As a preface to the detailed description, it should be noted that, as used in this specification and the appended claims, the singular forms “a”, “an” and “the” include plural referents, unless the context clearly dictates otherwise. Some general definitions are provided for the terms used herein. “Biofilm” is any group of microorganisms in which cells stick to each other on a living or non-living substrate. These adherent cells are frequently embedded within a self-produced matrix of extracellular polymeric substance (EPS). Microbes in a biofilm state make collective decisions by communicating with chemical signals called “quorum sensing.” The microbial cells growing in a biofilm are physiologically distinct from planktonic cells of the same organism, which, by contrast, are single-cells that may float or swim in a liquid medium. “Organosilane” means a compound of the family of compounds comprising the elements of silicon, oxygen, and carbon with a C—Si covalent bond and a nitrogen atom in a quaternary ammonium configuration. “Organosilane” also includes any quaternary ammonium salt of an organosilane. “Microbial cell” and “microbe” are used interchangeable and are understood to mean any single-celled organism. “Microcapsule” refers to a subset of the broader category of “microparticles,” wherein the microcapsule is a microparticle having a core comprising one material or compound surrounded by a distinctly different second material or compound. As the generally accepted size range for microparticles, a microcapsule has a size within the broad range of 1 micron to 1000 microns (1 millimeter). Therefore, the size range of a microcapsule, for the purposes of this application, is between that of a large nanoparticle to an object visible to the eye without magnification. Microparticle may also refer to a solid compound comprising the particle that is, itself, coated with the organosilane for purposes of becoming imbedded in a conditioning layer or more mature biofilm. “Substrate” means the skin and subcutaneous tissues of vertebrates, including the skin of ear canals, on which microorganism(s) are attached.

Disclosed is an antimicrobial product 100. Product 100 is an organosilane 102 in combination with other compounds in a mixture chosen according to the intended application of product 100.

The antimicrobial action of product 100 is provided by the organosilane. An organosilane is a molecule comprising a silicone atom covalently bonded to carbon. Organosilanes in general may be amphiphilic, having both water-soluble and lipid soluble components. Organosilane 102 comprises a hydrophilic “cap” comprising a silicon-tri-methoxy or silicon-tri-hydroxy “head,” and a hydrophobic “tail” comprising an eighteen or twenty-atom linear carbon chain. The head and tail are joined at a nitrogen atom bonded with two additional methyl groups to create a (cationic) quaternary ammonium group. The methoxy or hydroxy head groups facilitate enzymatically or chemically binding the organosilane to a substrate 140. The hydrophilic quaternary ammonium group allows for electrostatic attraction between the negatively-charged molecular species unique to the external cell walls of common to most bacteria and fungi. Once bound, the linear hydrophobic hydrocarbon tail of the organosilane traverses the phospholipid cell membrane, mechanically piercing and disrupting the membrane, causing lysis with death of the cell. This microbial killing mechanism is advantageous for several reasons. Organosilane 102 is not altered or consumed by its interaction with the targeted microbe. The organosilane is non-toxic and will not adversely impact the environment.

In various embodiments of the invention, other compounds are added to product 100. For some embodiments wherein product 100 is used in healthcare and veterinary medicine application, an anti-inflammatory 103 is added to reduce inflammation and itching. In some embodiments, a topical anesthetic is added to reduce pain from inflammation. For some embodiments wherein product 100 is used to kill microorganisms in a biofilm product 100 further comprises a cellulase enzyme. In some embodiments, product 100 further comprises other enzymes or compounds to interfere with quorum sensing utilized by microorganisms growing in a biofilm. In some embodiments, product 100 comprises an agent to modify viscosity. In some embodiments, product 100 comprises an agent to promote trans-epithelial delivery of un-bound product 100 through skin, keratin, or mucosal substrates. For some embodiments a keratolytic is added to destroy elements of the biofilm.

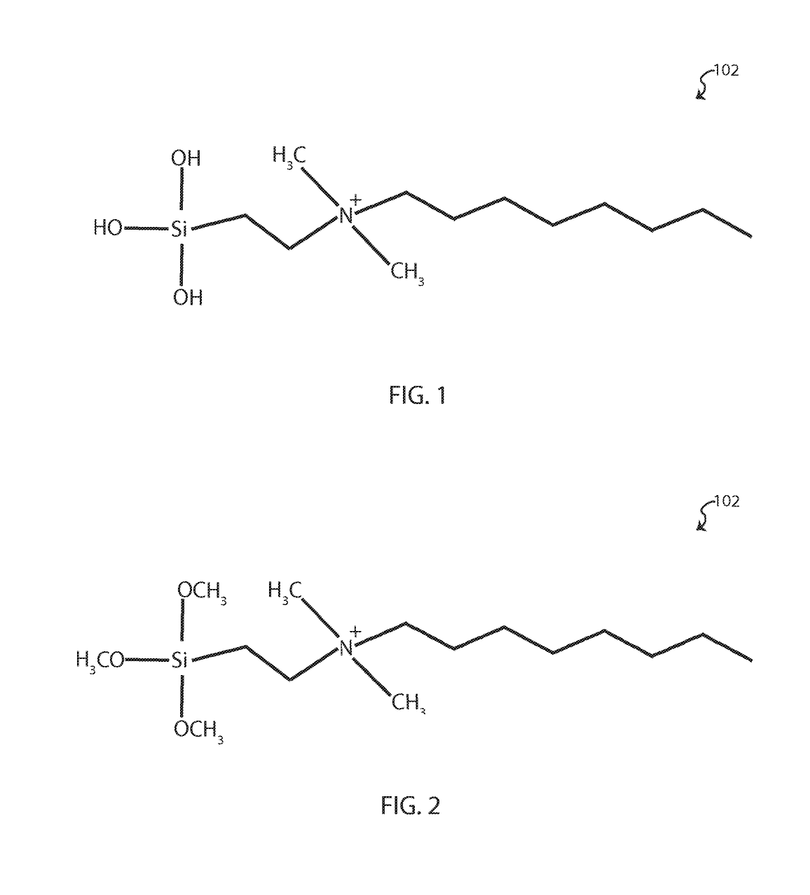

Referring to the drawings, FIG. 1 and FIG. 2 each depict an organosilane 102. These non-limiting examples show the fundamental structure of two organosilanes 102 with antimicrobial activity. Product 100, in some embodiments, may comprise an organosilane 102 with alternative molecular structures. Common to organosilanes 102, however, are a silyl “head,” a quaternary ammonium group, and an aliphatic hydrocarbon “tail.” Embodiments of product 100 comprise organosilane 102 and, in some embodiments, additional structural and functional components that complement one another to add functionality and performance to product 100, the structure and function of which will be described in greater detail herein.

In the example embodiment shown in FIG. 1, organosilane 102 is a 3-hydroxysilyl organosilane. The silyl “head” of the molecule is shown to the left of the figure, comprising three hydroxyl groups which, in some embodiments, are reacted to covalently bond with a biological or non-biological substrate. The quaternary ammonium group is also shown, connecting the silyl “head” with the aliphatic hydrocarbon “tail.” In the example embodiment shown in FIG. 2, organosilane 102 is a 3-methoxysilyl organosilane. In some embodiments, organosilane 102 is s 3-(trihydroxysilyl) propyl dimethyl octadecyl ammonium molecule. In some embodiments, organosilane 102 is a 3-(trimethoxysilyl) propyl dimethyl octadecyl ammonium molecule. In some embodiments, organosilane 102 is s 3-(trihydroxysilyl) propyl dimethyl dodecyl ammonium molecule. In some embodiments, organosilane 102 is a 3-(trimethoxysilyl) propyl dimethyl dodecyl ammonium molecule. A difference in ionic charge, the positive being the organosilane Nitrogen atom and the negative being the cell walls of most microbes, causes attraction and binding to the cell wall and damage to the cytoplasmic membrane, and further it is believed that mechanical killing of microbial cells 135 occurs by penetration and physical disruption of the microbial cell wall and phospholipid cell membrane by the hydrophobic “tail” of organosilane 102. Microbial cells 135 are drawn to a treated substrate 140 covered with adherent organosilane 102 molecules electrostatically by the cationic quaternary ammonium groups of organosilane 102. Amphiphilic quaternary ammonium compounds, including but not limited to organosilane 102, effect microbial killing by acting as detergents, wherein the cationic N+ atom ionically binds to negatively charged sites on lipopolysaccharides and constituent proteins of the bacterial cell wall, bringing the hydrophobic organosilane “tail” into proximity with the phospholipid cell membrane. In some embodiments, addition of other detergents to product 100 facilitate penetration of organosilanes 102 into subsurface layers of microbial biofilms by disrupting the complex mixed hydrophilic/hydrophobic layers of the biofilm. Some non-limiting examples of detergents added to product 100, in some embodiments, include other quaternary ammonium compounds, benzalkonium chloride, benzethonium chloride, and sodium oxychlorosene.

In some embodiments, antiseptic compounds are added to product 100 to act as biocidal adjuncts to organosilane 102. A few non-limiting examples of such compounds include hypochlorous acid, poloxamer iodine complex, iodine complex, secondary amyltricresols, hexylresorcinol, methyl resorcinol, and ethyl alcohol. In some embodiments, antibiotic compounds are added to product 100, particularly wherein product 100 is formulated to treat a biological substrate 140. Antibiotics, which may act topically and/or be systemically absorbed, are useful in treating invasive infection which may be present concurrently with a biological substrate 140 colonized with microbial cells 135, such as commonly occurs with bacterial or fungal otitis externa in dogs, cats, other mammals, and humans. Some non-limiting examples of antibiotic molecules which product 100 comprises, in some embodiments, include aminoglycosides, such as gentamycin or tobramycin; macrolides, such as erythromycin and azithromycin; flouroquinolones, such as ciprofloxacin and levofloxacin; polypeptides, such as polymixin B; and sulfonamides, such as sulfamethoxazole.

In some embodiments involving treatment of a substrate 140 wherein an invasive microbial infection is present with product 100, an anti-inflammatory compound is a useful therapeutic adjunct. Invasive microbial infection normally creates an inflammatory response. Inflammation creates local swelling, increases pain and/or itching, and, if marked may interfere with healing. This is particularly important in veterinary application wherein inflammation compels the animal to scratch at the infected substrate, aggravating inflammation and leading to additional irritation and more scratching. Therefore, treatment with a topical or systemic anti-inflammatory compound is often useful. In some embodiments, product 100 further comprises an anti-inflammatory molecule. Some non-limiting examples of such anti-inflammatory compounds include steroids, such as triamcinolone diacetate, hydrocortisone, beta methasone valerate, and beta methasone diproprionate. In some embodiments, product 100 further comprises a topical anesthetic to treat the pain and itching associated with the inflammatory response, some non-limiting examples including lidocaine hydrochloride, benzocaine hydrochloride, hexylresorcinol and methyl resorcinol.

### EXPERIMENTAL EXAMPLES

In one experimental example, a formulation of product 100 comprising organosilane 102 was successfully used to treat a severe long-standing case of a mixed bacterial/fungal infectious otitis externa and infectious dermatitis in a Shih Tzu dog. “Conner,” a neutered male Shih Tzu, aged 11 years 7 months, presented for re-evaluation of bacterial and monilial dermatitis (Malassezia sp.). Conner had a past history of keratoconjunctivitis sicca, generalized demodicosis, and allergies. The evaluation and subsequent treatment with product 100 was begun two months after failed conventional therapy with oral antibiotics (amoxicillin/clavulanate (Clavamox® 13.6 mg/kg) orally b.i.d. for four weeks) and an oral antifungal (fluconazole, 5.4 mg/kg once daily for two weeks; then once every-other-day for two additional weeks.) The earlier failed therapy also consisted of bathing the animal 2-3 times a week using an anti-seborrheic shampoo (KeratoLux®) and an antimicrobial shampoo (Duoxo Chlorhexidine® shampoo) followed by an oatmeal-based cream rinse (Episooth ®). The Conner's chest, neck, paws, and face were cleaned and treated twice daily with antimicrobial wipes (Douxo Chlorhexiding pads®) and an antimicrobial lotion (ResiKetoChlor®). The ears were cleaned once daily with a tris-EDTA/ketoconazole solution (TrizUltra plus Keto®) and treated twice daily with an amikacin otic preparation.

Dermatologic examination revealed extremely abundant purulent exudate in both ears with stenotic canals and erythema, lichenification, and edema on both medial pinnae. The animal had generalized mixed hypotrichosis/alopecia, erythema, hyperpigmentation, and lichenification with crusting over dorsal trunk, legs, paws, and ventrum. Hair on the face and neck was severely matted. The skin underlying the mats was crusted, with moist dermatitis and brown purulent exudate. Lymph node enlargement was palpated in the mandibular, prescapular, and popliteal node groups.

Cytological examination of skin scrapings revealed abundant bacterial dermatitis with cocci and diplococci. A generalized severe Malassezia (yeast) dermatitis was concentrated mostly on ventrum and paws. Otic examination revealed bilateral severe bacterial otitis externa with cytological examination revealing abundant mixed population of rods and cocci, along with occasional Malassezia. Demodex canis was seen in all life stages. Fine-needle aspirates of peripheral lymph nodes (left prescapular and left popliteal) were consistent with reactive lymphadenopathy.

The otitis externa was treated initially by cleaning the ears with a salicylic acid based ear cleaner (Otoclean®) and instillation of 0.5 ml of product 100 comprising organosilane 102 (3-(trihydroxsily) propyldimethyloctadecyl ammonium chloride) in each ear. Mats were removed and the dog was bathed and groomed over two days using (Splash plus® shampoo), followed by antimicrobial shampoo (Douxo Chlorhexidine® shampoo), followed by essential fatty acid cream rinse (Hylyt® cream rinse). After bathing, product 100 comprising organosilane 3-(trihydroxsily) propyldimethyloctadecyl ammonium chloride was applied to facial folds and over body using moistened gauze sponge pads.

“Conner” returned 7 days later for a brief recheck. Otic cytology revealed complete resolution of the bilateral bacterial otitis externa and significant improvement of the bacterial dermatitis at all sites. Only a mild amount of ceruminous exudate was observed in each ear. Lichenification and moist dermatitis had decreased substantially. No purulent exudate was observed at any site. Topical therapy was repeated (bathing with Splash plus® shampoo, Douxo Chlorhexidine® shampoo, and Hylyt® cream rinse) followed by application of product 100 comprising organosilane 102 (3-(trihydroxsily) propyldimethyloctadecyl ammonium chloride) using moistened gauze pads. Otic therapy was repeated with a cleaning using salicylic acid based ear cleaner (Otoclean®) and treated by instilling product 100 comprising organosilane 102 (3-(trihydroxsily) propyldimethyloctadecyl ammonium chloride) in each ear. Weekly rechecks over the course of one month showed continued improvement and no recurrence of bacterial or Malassezia infections.

In a second experimental example, “Lillie,” a fawn Puggle born 4/2009, was seen for an acute episode of otitis in her left ear in November 2014 after no previous history of any trauma, bathing, swimming, or medical issues. A purulent discharge was exuding from the ear canal and inflammation was apparent in the aural pinna on both the anterior and posterior aspect. A culture swab was collected for Clinpath® ID and sensitivity and product 100 comprising organosilane 102 (3-(trihydroxsily) propyldimethyloctadecyl ammonium chloride) solution with steroid was directed to be flushed daily into the ear canal after the ears were cleaned utilizing a Q-tip soaked in a general cleansing solution.

Results of the culture and sensitivity showed a mixed infection of Pasteurella Multocida 1+, coagulase positive Staph species 3+, and Malassezia yeast 1+. Flush medication was continued for two weeks and the ear was reexamined. The external auditory canals were wide open and showed no purulent debris or inflammation indicative of any previous infection. General cleaning on a regular basis was recommended with Epi-Otic®.

After 1 month, Lillie returned with a second bout of otitis occurring post grooming. It was learned that the groomer had been applying a medication in the ears for “ear mites” and the infection started within 48 hours post application. The owner was again instructed to flush the ears with product 100 comprising organosilane 102 (3-(trihydroxsily) propyldimethyloctadecyl ammonium chloride) solution including a steroid and have them rechecked in 1 week. Within 1 week the owner called back to report that the ears had cleared up and that she would not need the recheck appointment.

Lillie has since been seen for underlying allergies and has been itching at her ears causing sores and lesions on the external pinna. Otic cytology revealed no etiologic agent, just inflammatory and epithelial cells. Although the generalized erythema has become more prevalent on Lillie's body, her ears have been able to remain infection free to this point with weekly routine cleansing and flushing with the product 100 solution.

Some antibiotics and enzymes function optimally within a relatively narrow pH range. Accordingly, some embodiments of product 100 add a buffer to the treating compound at concentration levels sufficient to maintain the pH range required for optimal activity of the components of the product. The particular buffer is selected based upon the local conditions present on the biological substrate 140. Buffers to maintain ambient pH within a desired range include, but are not limited to, citrates, sulfonates, carbonates, and phosphates. The preferred buffering compound and concentration of same useful for maintaining a desired pH range are dependent on ambient micro-environmental conditions at the treated area and known to those skilled in the art.

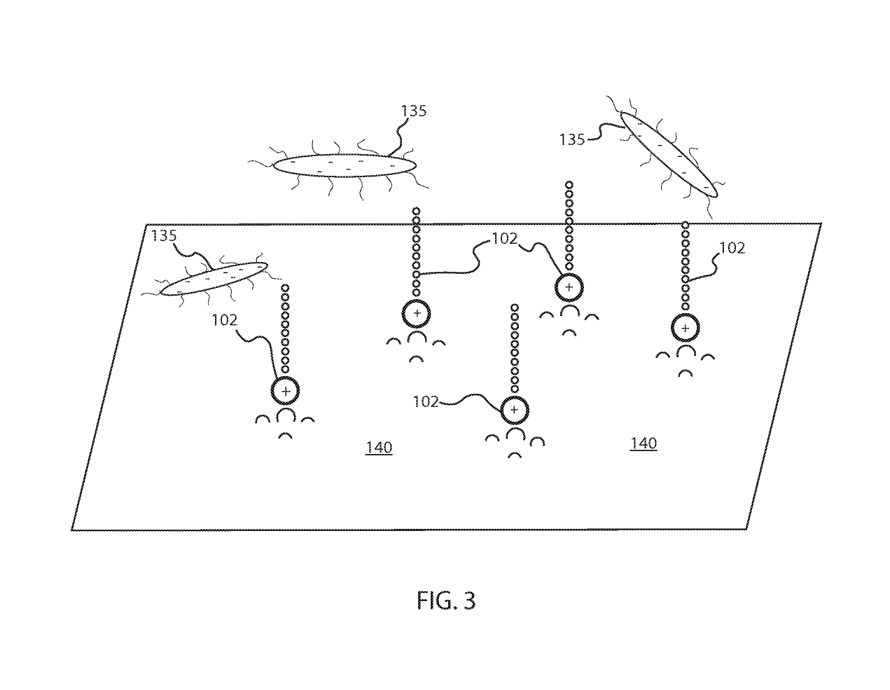

FIG. 3 is a diagram showing organosilane 102 molecules bonded to a substrate 140 in the presence of a microbial cell 135. Microbial cells 135 may be bacteria (as shown in FIG. 3), archaebacteria, protists, or fungi. A microbe generally carries a negative net charge at the cellular substrate due to constituent membrane proteins. For example, the cell walls of Gram-positive bacteria contain negatively-charged teichoic acids. The cell membranes of Gram-negative and Gram-positive bacteria (and other microbes) comprise negatively charged phospholipids molecules. The negatively-charged substrates of free-floating “planktonic” microbes, therefore, are electrostatically attracted to cationic compounds, such as the quaternary ammonium group-containing organosilane 102 coating substrate 140, and may bind to the cell wall and cytoplasmic membrane. If the compound, such as organosilane 102, is amphiphilic, the hydrophilic portion of the molecule may traverse both the bacterial cell wall and cytoplasmic membrane, causing cellular lysis and death of microbial cell 135. As a result, the attachment and bonding of product 100 comprising organosilane 102 to substrate 140 results in substrate 140 becoming configured to kill microbial cells 135 on contact. Because this substrate killing does not disrupt and consume product 100, frequently repeated application is not required and microbial killing is accomplished without releasing a biocide to the environment.

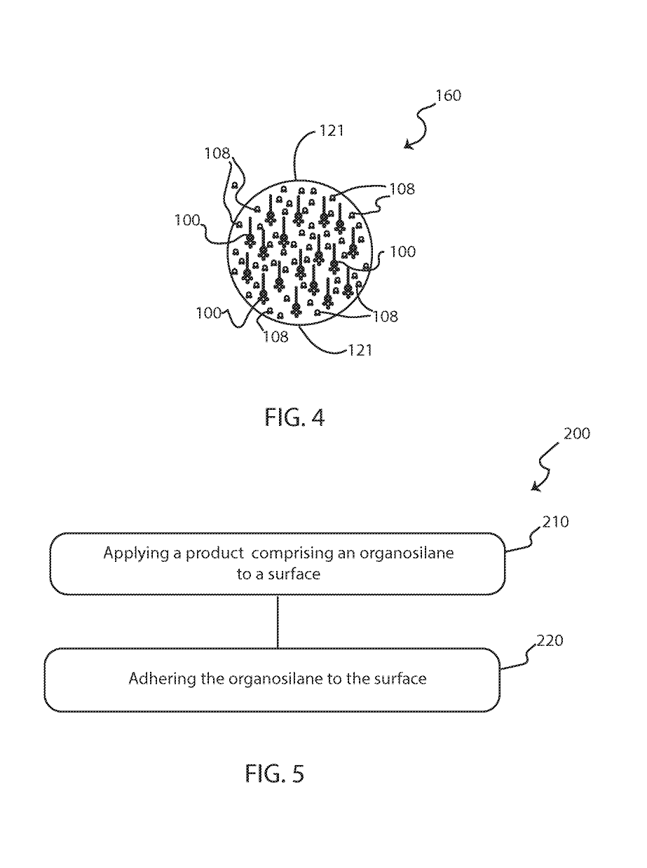

Product 100 additionally comprises a carrier 108, schematically shown in FIG. 4. Carrier 108, in some embodiments, is a compound that holds the various sub-components of product 100 in suspension or solution. The specific compound used is chosen based upon the characteristics necessary for the end-use application of product 100. For example, if product 100 is to be used on a substrate, such as skin lining the external auditory canal of a dog, carrier 108 may be an emollient, wax, alcohol, non-ionic surfactant, or other suitable compound. Non-limiting examples include excipients such as cetyl alcohol, tyloxapol, methyl paraben, polyethylene glycol, coconut oil, or cottonseed oil. The carrier is, in some embodiments, be employed to form product 100 into a gel, lotion, ointment, liquid solution, or liquid suspension, according to the intended end-use of product 100.

The concentration of organosilane 102 by weight of product 100 is also selected according to the desired end-use of product 100. In situations where high antimicrobial activity is needed for treating a well-established biofilm, concentrations of organosilane provide a higher density of adherent organosilane molecules on substrate 140. In effect, the “forest” of aliphatic hydrocarbon molecular “tails” is thicker. Additionally, higher organosilane concentrations create a higher cationic charge density, resulting in both stronger electrostatic microbial attractive forces and detergent effects on the microbial phospholipid cell membrane. Because some organosilane molecules become separated from substrate 140 with exfoliation, dressing changes, and cleaning, a higher concentration of organosilane 102 in product 100, in some embodiments, allows product 100 to act as a substrate antiseptic for a longer period of time. Concentrations of organosilane 102 in product 100 of up to and over 5% by weight may be used, however, when used in concentrations of over about 3%, polymerization of organosilane 102 within product 100 prior to application on substrate 140 increases through intermolecular cross-linking via —S—O—S— covalent bonds. In applications to substrates, such as a cutaneous epithelium or an open wound treated using product 100, product shedding through epithelial turnover may requires frequent re-application of product 100, in some applications. The risk of bacterial and other microbial resistance to an antimicrobial compound, regardless of the mechanism of action of the compound, theoretically increases with increasing environmental encounters between bacteria and other microbes, and the antimicrobial compound. It is prudent, therefore, to strive to minimize the amount of any composition with antimicrobial activity within the general environment. Accordingly, in the aforementioned and other situations wherein frequent re-application of product 100 is necessary, lower concentrations of organosilane 102, down to and below 0.1by weight in product 100, are useful by lowering the overall amount of organosilane 102 ultimately discharged into the environment. Notwithstanding the theory, it is believed that the risk to the environment and/or causing biofilm mutations by use of these formulations is minimal.

Advantages of product 100 according to the several embodiments described herein, include the aforementioned non-leaching properties of product 100; decreased risk of microbial resistance giving the unique mechanical disruption of cell walls and cell membranes common to all microbes; the relative non-toxicity of organosilanes when used to treat a biological substrate infection; and the stability of product 100 bonded to substrate 140 decreasing the need for frequently repeated application and subsequent dispersal of organosilanes and other constituent compounds of product 100 into the environment.

In some embodiments, product 100 and/or delivery system 160 is applied to an existing biofilm. Product 100 comprising organosilane 102 has amphiphilic properties and penetrates an existing biofilm, bringing the biocidal organosilane 102, along with additional antibiotic and/or antiseptic compounds in some embodiments, to deeper layers of an existing biofilm, killing microbial cells 135 within the extracellular biofilm matrix and disrupting the biofilm. In some embodiments, product 100 is applied as an aerosol, other spray, or wiped onto substrate 140. In some embodiments, substrate 140 with existing microbial contamination, with or without an associated biofilm, is treated by applying liquid product 100. One non-limiting example is wherein product 100 is applied in liquid form to a well-established biofilm within the chronically-infected external auditory canal of a dog, as in the case study described herein above. In some embodiments, the treated article is placed on the skin, mucosa, open wound substrate, or other biological substrates of humans, animals, and/or other living organisms. In these instances, the positive negative electrical attraction between the shells of the cells and the formulation in the treated article pulls the cells out of the infected area into the article which can then be disposed in a safe manner. In some embodiments, the method further comprises a step placing treated material in proximity to an area of microbial colonization. In some embodiments, the delivery system is a microcapsule.

Because in some embodiments, product is a non- leaching compound that is bound to substrate 140 of an article 142, the area may be treated without ever placing or applying the antimicrobial product directly into the area 24 or in physical continuity with the area 24, if desired. In some embodiments, treated article 142 is placed in contact with an open wound to treat the wound. In some embodiments, the treated article is inserted within a semi-enclosed body region, such as the external auditory canal or nares to treat infection in these regions known to be conductive to pathogenic microbial growth. In some embodiments, the treated article is placed on the skin, mucosa, open wound substrate, or other biological substrates of humans, animals, and/or other living organisms. In these instances, the positive negative electrical attraction between the shells of the cells and the formulation in the treated article pulls the cells out of the infected area into the article which can then be disposed in a safe manner.

Some embodiments of product 100 further comprise proteolytic and other enzymes as components useful in disrupting an established biofilm. Expansion of colonies of biofilms on substrates, such as the skin lining the external ear canal of a human or other mammal, is enhanced by the process of constant desquamation. Biofilms in this and other examples are disrupted by incorporation of enzymatic adjuncts, such as any one of the keritinase family of proteolytic enzymes. A few non-limiting examples include incorporation of collagenase, cellulase, or keritinase into product 100. In addition to proteolytic keratinases, some embodiments of product 100 comprise other enzymes. For example, N-acyl homoserine lactone is a bacterially-produced amino sugar acting as a hormone involved in quorum sensing. Some actions of N-acyl homoserine lactone include bacterial self-limitation of microbial population density and other population-based characteristics, such as gene regulation of enzyme systems and the expression of flagella versus pili. Enzymes acting upon an N-acyl homoserine lactone substrate destroy and substrate and thereby disrupt bacterial signaling systems in a biofilm, acting as an adjunct to proteolytic keratinases and other components of product 100, in some embodiments, to disperse existing biofilms and interfere with new biofilm formation.

FIG. 4 shows a microcapsule 121 encasing product 100. Microcapsule 121 is one example of delivery system for product 100. Microcapsule 121, in some embodiments, comprises a material enveloping and containing product 100. Non-limiting examples of compounds used to form microcapsule 121 include polyvinyl alcohol, cellulose acetate phthalate, gelatin, ethyl cellulose, glyceryl monostearate, bees' wax, stearyl alcohol, and styrene maleic anhydride. Many other compositions of microcapsule 121 are possible, and the exact composition, construction, and manufacture of microcapsule 121 is chosen from the broad range of compositions and manufacturing techniques for microcapsules generally, and which are readily available and known to those skilled in the art. In the example delivery system shown in FIG. 4, liquid product 100 is encapsulated within microcapsule 121 and thereafter released when microcapsule 121 is broken. Breakage of microcapsule 121 is effected at a chosen time and in a manner specific to the particular use of product 100. For example, microcapsule 121 may be broken by scratching, as when a dog scratches the ostium of its external auditory canal in response to itching arising from inflammation. In this manner, product 100 is configured to remain on substrate 140. Because product 100 becomes active upon breaking of microcapsule 121, the effective useful life of product 100 begins.

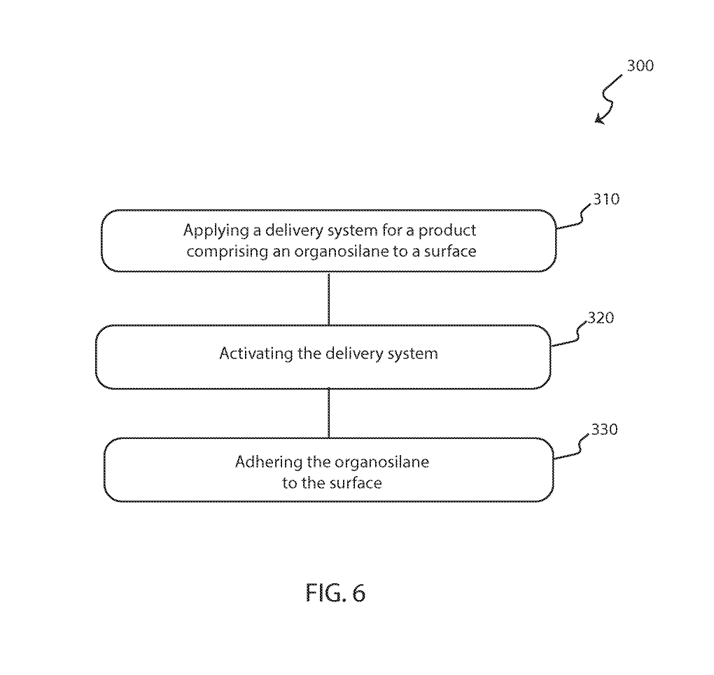

FIG. 6 shows a method of treating infection and/or infectious disease, as does FIG. 5. Applying step 310 of method 300 (step 210 of method 200 of FIG. 5) comprises applying a delivery system for a product comprising an organosilane to a substrate. In some embodiments, the delivery system of applying step 310 (step 210 of FIG. 5) comprises a microcapsular delivery system, such as microcapsule 121 discussed herein above. In some embodiments, the delivery system of applying step 310 (step 210 of FIG. 5) is a delivery system not comprising microcapsules such as by drops or spraying the area to be treated. Activating step 320 of method 300 comprises activating the delivery system. In some embodiments, activating step 320 comprises breaking microcapsules encasing the product comprising the organosilane. In some embodiments, breaking microcapsules occurs when a dog or other animal scratches the treated substrate in response to itching caused by inflammation. Adhering step 330 of method 300 (step 220 of FIG. 5) comprises adhering the organosilane to the substrate. In some embodiments, adhering step 330 (step 210 of FIG. 5) involves reaction of released, activated components of the product, such as the organosilane for example, to the material composition of the substrate. For example, the organosilane adheres to protein molecules such as keratin, collagen, and other proteins located on the skin surface or within the intercellular matrix of subcutaneous and deeper tissue. Bonding of the organosilane to the substrate may be by covalent bonding, ionic bonding, electrostatic bonding, or other interaction between the organosilane and the substrate.

Method 200 and/or method 300 may further comprise placing a treated article into a location prone to microbial growth, such as an area where microbes are known or expected to exist, and/or an area 24 where biofilms have formed or may develop. A treated article is a non-biological object comprising a substrate to which a product comprising an organosilane has been applied and adhered. Some non-limiting examples of a treated article include a gauze plug inserted in the ostium of an external auditory canal, and a gauze pad or other dressing material placed upon an open wound. By placing the treated article into close proximity but not necessarily in direct contact with such an area, the electrostatic properties of the product comprising an organosilane or and/or additional cationic detergent or other substance may attract and draw nearby microbes to the cationic product, thereby reducing the concentration of microbes in the adjacent area sought to be protected from microbial colonization and/or infection and possible biofilm formation.

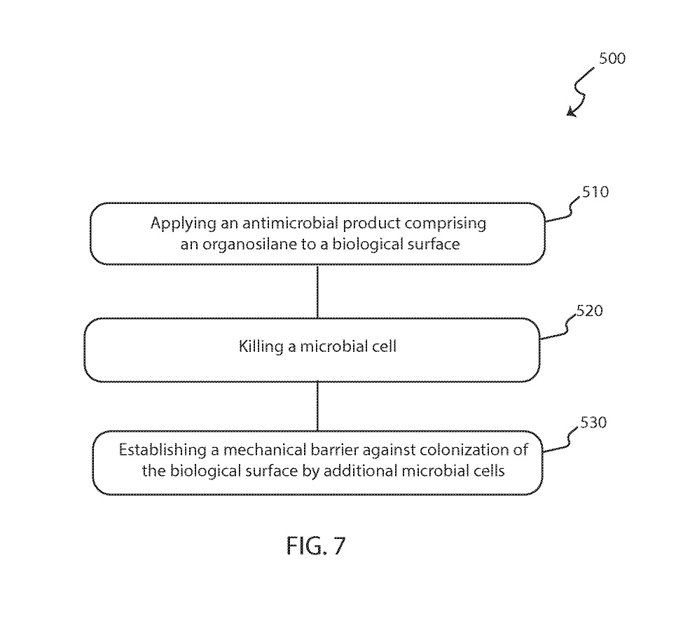

FIG. 7 shows a method 500 of treating and preventing an infection on a substrate. Method 500 comprises an applying step 510, a killing step 520, and an establishing step 530. Applying step 510, in some embodiments, comprises applying an antimicrobial product comprising an organosilane to a substrate. The substrate, in some embodiments, is a site of invasive local infection and may include a high density of bacterial, fungi, and/or other microorganisms. Killing step 520 comprises the killing of microbial cells via the mechanism(s) of action of the antimicrobial product. Mechanisms of killing include mechanical disruption of the cell wall and/or cell membrane by an organosilane molecule, ionic disruption of the cell membrane by cationic (or anionic) detergents, or by other metabolic disruptions of microbial gene expression, protein synthesis, metabolism, and other microbial cellular processes by various constituent antibiotics present in some embodiments of the antimicrobial product. These aforementioned and other mechanisms of microcidal activity of the antimicrobial product may occur singularly or in any combination, according to some embodiments of the invention. Establishing step 530 comprises establishing a mechanical barrier against colonization of the biological substrate by additional microbial cells not initially present on the biological substrate. In some embodiments, establishing step 530 arises through the action of adherent organosilane molecules presenting an expanse of hydrophobic aliphatic hydrocarbon molecular “tails,” which present a direct mechanical barrier for additional microorganisms seeking to adhere to and colonize the biological substrate via the mechanisms discussed at length herein above.

In some embodiments, method 500 additionally comprises a step penetrating a biofilm. The penetrating step is occurs through the action of the product comprising compounds that penetrate and disrupt a biofilm, such as cationic or anionic detergents; enzymes, including cellulase and other protein hydrolases in particular, keratolytics including salicylic acid, and N-acyl homoserine lactone-lactonase.

Exceptional results can be obtained with the antimicrobial compounds with antiseptic and antibiotic activity; in particular, to organosilane compounds for treatment of human and animal infection, creating and antiseptic coatings for substrates, and methods of using the same disclosed in this description of several embodiments of the invention. The disclosed product provides a durable treatment of a substrate, minimizes leaching of antimicrobial into the environment, minimizes opportunities for development of microbial resistance due to its combined mechanical and electrostatic mechanisms of action, is safe and effective in treating resistant invasive infections of the skin, toenails, hooves and other biological substrates, and may be incorporated directly into articles such as bandages and wound coverings by way of a delivery system.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above.

What is claimed is:

1. A method to treat otitis externa caused by a microbial organism in an animal or human in need thereof, comprising administering to the external auditory canal of the animal or human a microparticle coated on the external surface with an effective amount of 3-(trihydroxysilyl)propyldimethyloctadecyl ammonium chloride.

2. The method of claim 1, wherein the microparticle is composed of a compound selected from cellulose acetate phthalate, ethyl cellulose, glyceryl monostearate, bees wax, stearyl alcohol, polyvinyl alcohol, and styrene maleic anhydride.

3. The method of claim 1, wherein the microbial organism is a bacterium.

4. The method of claim 3, wherein the bacterium is a gram-negative bacterium.

5. The method of claim 3, wherein the bacterium is a gram-positive bacterium.

6. The method of claim 1, wherein the microbial organism is a fungus.

7. The method of claim 1, further comprising administering an effective amount of an additional solution of 3-(trihydroxysilyl)propyldimethyloctadecyl ammonium chloride by drops or spray to the external auditory canal of the animal or human.

8. A method to treat otitis externa caused by a microbial organism in an animal or human in need thereof, comprising administering to the external auditory canal of the animal or human a microcapsule, wherein the microcapsule encapsulates a solution comprising an effective amount of 3-(trihydroxysilyl)propyldimethyloctadecyl ammonium chloride in water.

9. The method of claim 8, wherein the microcapsule is composed of a compound selected from cellulose acetate phthalate, ethyl cellulose, glyceryl monostearate, bees wax, stearyl alcohol, polyvinyl alcohol, and styrene maleic anhydride.

10. The method of claim 8, wherein the microbial organism is a bacterium.

11. The method of claim 10, wherein the bacterium is a gram-negative bacterium.

12. The method of claim 10, wherein the bacterium is a gram-positive bacterium.

13. The method of claim 8, wherein the microbial organism is a fungus.

14. The method of claim 8, further comprising administering an effective amount of an additional solution of 3-(trihydroxysilyl)propyldimethyloctadecyl ammonium chloride by drops or spray to the external auditory canal of the animal or human.

15. An antimicrobial product comprising a microparticle coated on the external surface with 3-(trihydroxysilyl)propyldimethyloctadecyl ammonium chloride in a carrier.

16. The antimicrobial product of claim 15, wherein the microparticle is composed of a compound selected from cellulose acetate phthalate, ethyl cellulose, glyceryl monostearate, bees wax, stearyl alcohol, polyvinyl alcohol, and styrene maleic anhydride.

17. The antimicrobial product of claim 15, wherein the carrier is selected from cetyl alcohol, tyloxapol, methyl parabens, polyethylene glycol, coconut oil, and cottonseed oil.

18. The antimicrobial product of claim 15, further comprising an enzyme.

19. The antimicrobial product of claim 18, wherein the enzyme is a proteolytic hydrolase enzyme.

20. The antimicrobial product of claim 18, wherein the enzyme acts upon a substrate comprising N-acyl homoserine lactone.
